(19) United States
(12) Patent Application Publication
(10) Pub. No.: US 2009/0311197 A1
(43) Pub. Date: Dec. 17, 2009

(54) Method and Compositions for Oral Hygiene

(75) Inventors: Radek Romanowski, Komoka (CA); Peter Emily, Lakewood, CO (US); Stan Alkemade, Arva (CA)

Correspondence Address: JOHN S. PRATT, ESQ;KILPATRICK STOCKTON, LLP, 1100 PEACHTREE STREET, SUITE 2800, ATLANTA, GA, 30309, US

(73) Assignee: Imrex, Inc., Komoka (CA)

(21) Appl. No.: 12/509,768

(22) Filed: Jul. 27, 2009

Related U.S. Application Data
(62) Division of application No. 11/018,851, filed on Dec. 21, 2004, now abandoned.
(60) Provisional application No. 60/532,303, filed on Dec. 22, 2003.

(51) Int. Cl. A61K 9/68; A61K 8/66; A61Q 11/00

(52) U.S. Cl. 424/48; 424/50

(57) ABSTRACT

The present invention comprises novel compositions and methods for oral hygiene and for treating and preventing oral disease in humans and in animals. In one embodiment, the novel compositions of the present invention comprise a unique oral hygiene solution that can be added to drinking water. The invention provides compositions and methods for maintaining oral health that are convenient to use and are formulated so that they are safe for regular use by humans and animals.

### CROSS-REFERENCE TO RELATED APPLICATIONS

The present application claims priority to U.S. Provisional Application Ser. No. 60/532,303 filed on Dec. 22, 2003.

### FIELD OF THE INVENTION

The present invention is related to methods and compositions for oral hygiene. In particular, the present invention includes novel compositions for prevention and treatment of oral disease and for maintaining oral health.

### BACKGROUND OF THE INVENTION

Maintenance of good oral health and prevention of oral disease is a necessity for both humans and animals. Unlike animals, including animals that are pets, humans have the ability to exercise control over oral and dental hygiene by regular cleaning and regular dental visits. Even so, humans still experience oral problems ranging from cavities to more severe cases of gum disease. The need for proper oral care is just as important for animals, but for a variety of reasons, is harder to meet.

Most veterinarians recommend an oral hygiene program that includes brushing an animal's teeth on a regular basis with a dentifrice specially formulated for animals, not human toothpaste or baking soda. Human toothpastes are often made with various foaming agents that are intended to be rinsed, not swallowed. Even occasional consumption of these toothpastes may cause stomach problems and most pets cannot be taught to rinse. The high sodium level of baking soda may cause problems in some animals, especially those with a heart condition. The same preventive techniques recommended for humans, especially routine brushing, are the most effective means of controlling plaque and tartar build-up in animals. However, as discussed above, even routine brushing of a pet's teeth is challenging for a variety of reasons for most pet owners.

Oral health problems in animals may be indicated by any one or a combination of the following symptoms: persistent bad breath, sensitivity around the mouth, pawing at the mouth, loss of appetite, plaque (often not visible unless stained), tartar (creamy-brown, hard material), calculus (mineralized tartar/plaque), bleeding, inflamed or receded gums, loose or missing teeth and difficulty eating and chewing food.

According to the American Veterinary Dental Society (AVDS), eighty percent of dogs and seventy percent of cats have periodontal (gum) disease by the age of three. Not only are these alarming statistics, it is even more noteworthy that proper dental care could increase the life of these animals by two to five years.

Dental care in dogs and cats has become quite common over the last six years. Like humans, dog's teeth and gums are also susceptible to many of the same oral health problems—gingivitis and periodontal disease. Unlike humans, animals rarely get cavities. This is because cavities are primarily caused by the high sugar content of the human diet. Periodontal disease affects both human and mammals alike. Periodontal disease is caused by bacteria and plaque that attach to the soft gum tissue of the mouth. The first stage of periodontal disease is gingivitis and is very common. In this stage, the bacteria have mixed with saliva and formed plaque. The plaque adheres to the teeth and hardens, forming tartar and calculus. These tartar deposits irritate the gum tissue and cause inflammation, swelling and infection. It is at this stage that gingivitis is most notable.

Early warning signs of gingivitis include sensitive gum tissue, redness or bleeding gums, trouble eating/chewing and bad breath. The breath may take on a sulfur (rotting eggs) odor from the by-products of the bacteria in the mouth. This is often the first sign of gingivitis and serious dental problems.

If caught at this stage, gingivitis is treatable. A thorough dental exam and cleaning most likely is needed. Many dogs will also most likely need to be put under anesthesia. (This presents its own series of side effects and dangers as well.) If gingivitis is not treated, it will progress to periodontal disease. Periodontal disease is not treatable. At this stage, there is irreversible bone loss and tooth damage. Roots are also weakened and the animal may experience loose teeth and teeth that simply fall out. Animals may also begin to lose weight. This can lead to other problems associated with improper nutritional intake. Dental treatment will be needed and may result in the extraction of teeth. Anesthesia is required for extraction of teeth, increasing risk to the animal. Additionally, the bacteria and infection in the mouth may spread through the bloodstream to cause heart, kidney or liver disease. These diseases can cause serious damage to the organs and lead to premature death.

Given the terrible, and in many cases irreversible effects, of periodontal disease, it is important to note that the effects are preventable. Veterinarians recommend instituting regular dental examinations and developing an at-home dental care program including a proper diet and mix of dry and wet foods. However, as acknowledged by pet care professionals, diet alone cannot prevent dental problems.

Regular cleaning for maintaining oral hygiene is necessary in order to avoid the oral and dental problems discussed above. Though tooth brushing is certainly an option, given the drawbacks already mentioned, a more practical option for many pet owners would be a method of administering effective oral hygiene compositions by way of an ingestible solution.

There are clear indications that oral health status has a profound effect on an animal's general health. Periodontal disease may cause bacteria and toxins to enter the bloodstream with potentially deleterious effects on internal organs. Conversely, poor systemic health may manifest in the oral cavity in various ways and may also exacerbate periodontal disease. An animal's dental examination is therefore not always limited to the oral cavity but frequently includes a general physical examination. Laboratory examinations, to evaluate systemic disease concerns, are also commonly performed. Some dogs and cats suffer from chronic oral infection or stomatitis, a poorly understood condition that is frustratingly difficult to treat.

Tumors account for another important group of oral problems in animals. Tumors of the mouth and throat are common in the dog but occur less frequently in the cat. Oral tumors frequently go unnoticed by pets' owners until the tumor reaches a fairly advanced stage of development, making it more difficult to treat successfully. A variety of lesions may occur, including benign and malignant conditions. Non-cancerous masses and swellings such as gingival hyperplasia and infectious conditions may be confused with oral tumors. Conversely, oral malignancies may present as non-healing, ulcerated sores instead of “typical” prominent masses.

What is needed therefore is an easy to use, effective system for maintaining good oral health as well as preventing and treating oral disease. What is needed are effective methods and compositions for maintaining good oral hygiene, as well as for preventing and treating oral disease in humans and in animals. Preferably such methods and compositions should be easy-to-use and comprise antimicrobial and anti-plaque agents. In addition, such methods and compositions should provide long-term effectiveness against oral problems including tartar build-up on teeth, gingivitis, and halitosis. Yet another desirable feature of such methods and compositions should be the resulting improved appearance of the oral cavity such as whiter teeth and healthy gums. Such methods and compositions should be affordable, safe and easy to use on a regular basis.

### SUMMARY OF THE INVENTION

The present invention solves the problems described above by providing novel compositions and methods for improving oral hygiene, for preventing and treating oral disease and for maintaining good oral health in humans and in animals. Unlike currently available products, the present invention provides unique methods and compositions that are safe and effective for regular use by both humans and animals. The present invention satisfies the long felt need for compositions that enable good at-home oral care for humans and animals.

The compositions of the present invention contain xylitol in combination with one or more other ingredients such as a surface adherent, thickener, emulsifier, preservative, breath freshener, antimicrobial, and a sweetener. Artificial color is optionally added. In another embodiment, the compositions of the present invention contain xylitol and a proteolytic enzyme, emilgase, in combination with one or more other ingredients such as a surface adherent, thickener, emulsifier, preservative, breath freshener, antimicrobial, and a sweetener. In one embodiment the compositions include antimicrobial and anti-plaque agents. In an alternative embodiment, the compositions further comprise antibiotics, antifungals, antiseptics, immunomodulators, immunostimulants and/or anticancer drugs known to one skilled in the art. In yet another embodiment, the compositions comprise taste-enhancing components that may be selected according to the user's preference.

Unlike the methods and products currently available, the present invention is effective in providing long-term improvements in oral care. Also unlike breath fresheners that rely on short-term exposure to antimicrobials and odor masks, the present invention is effective in correcting the underlying problem of oral disease and provides a more permanent solution to disorders such as halitosis.

A desirable effect of using the compositions of the present invention includes the improved appearance of the oral cavity including whiter teeth and healthy gums.

The present invention comprises a unique oral hygiene solution that can be added to drinking water or incorporated into various other objects that enter the oral cavity (including but not limited to food (such as biscuits and cookies), dental chew, candies, toothpaste, oral gels). This aspect of the invention makes it particularly useful for animals. The invention provides a method for maintaining oral health that is convenient to use and is formulated for animals so that the toxic side effects of human dental products are not an issue.

One of the main advantages of this invention is a high degree of animal caretaker (i.e. pet owner, kennel supervisor, barn manager, zookeeper) compliance. As the animal ingests the solution, the solution works to repel and retard plaque and eliminate the infectious organism and infectious organism by-products. In certain embodiments, the ingestible solution may take the form of a liquid or solid, and may optionally comprise an attractive flavor-enhancing component suitable for humans or animals.

The novel oral hygiene compositions of the present invention also provide advantages for humans. For example, the ease of administration together with pleasant tasting flavors makes the use of these compositions a highly desirable method of maintaining good oral hygiene, especially for young children, as well as the elderly or physically handicapped, who may not be able to brush their teeth very effectively. In addition, the compositions may easily be used in a variety of convenient embodiments including but not limited to gels, lozenges, and whitening molds.

Accordingly, it is an object of the present invention to provide compositions and methods for improving oral hygiene, and for preventing and treating oral disease.

It is another object of the present invention to provide compositions and methods for treating diseases and processes that are associated with the oral cavity.

It is yet another object of the present invention to provide compositions and methods for treating and preventing oral problems such as gingivitis, periodontal disease, stomatitis and halitosis.

It is another object of the present invention to provide compositions and methods for treating or repressing the growth of oral and throat cancer.

Another object of the present invention is to provide compositions and methods for prevention of tartar build-up on teeth.

It is another object of the present invention to provide compositions and methods for maintaining healthy teeth and gums.

Another object of the present invention is to provide methods and compositions for oral hygiene that are safe and easy for regular use by humans and animals.

Yet another object of the present invention is to provide novel compositions that may be added to regularly ingested food and beverages that impart a positive effect on oral hygiene.

Still another object of the present invention is to provide novel compositions that provide protection against oral infections.

Another object of the present invention is to provide novel compositions that optionally further comprise antimicrobials, antibacterials, antivirals, antifungals, antiseptics, immunomodulators and/or anticancer drugs that enable the prevention and treatment of oral disease and that enable the maintenance of good oral health.

It is yet another object of the present invention to provide novel compositions and methods that enable the cleaning and whitening of teeth.

Yet another object of the present invention is to provide novel compositions for maintaining good oral health further comprising flavor enhancing ingredients.

Another object of the present invention is to provide novel oral hygiene compositions and methods that have both a local and systemic effect.

Still another object of the present invention is to provide compositions and methods that maintain oral health by decreasing odor-causing germs.

Yet another object of the present invention is to provide compositions and methods that are available in easy-to-use forms such as powders that may be added to beverages such as drinking water, flavored waters, carbonated beverages, juices and similar such products.

It is another object of the present invention to provide compositions and methods comprising food and beverage additives that are non-toxic and are easily ingestible.

Another object of the present invention is to provide compositions and methods for improving oral hygiene in humans and in animals, such as dogs and cats, wherein such compositions comprise pleasant tasting additives that are incorporated into normally ingested food and water.

Yet another object of the invention is to provide compositions and methods for improving oral hygiene in humans and in animals such as dogs and cats, wherein such compositions comprise pleasant tasting additives that are incorporated into normally ingested food and water, and wherein such compositions optionally further comprise antimicrobials, antibacterials, antivirals, antifungals, antiseptics, immunomodulators and/or anticancer drugs.

Another object of the present invention is to provide compositions and methods for improving oral hygiene wherein such compositions can be incorporated into objects normally placed in the mouth such as chew toys, teething rings, or drinking utensils (such as feeding bottles for the young).

These and other objects, features and advantages of the present invention will become apparent after a review of the following detailed description of the disclosed embodiments and the appended claims.

### BRIEF DESCRIPTION OF THE FIGURES

FIG. 1(A-E) provides specific formulations for various embodiments of the of the oral hygiene compositions of the present invention (liquid formulas, powder formulas, and effervescent powder formulas).

FIG. 2 provides questions and verbal analogue scoring used in the study to evaluate the effectiveness of the oral hygiene compositions claimed herein (Example 2).

FIG. 3 provides a table of responses accompanying the study done to evaluate the effectiveness of the oral hygiene compositions claimed herein (Example 2).

### DETAILED DESCRIPTION

The present invention provides novel ingestible compositions that are useful in methods to improve oral hygiene in humans and animals.

### Compositions

The compositions of the present invention contain xylitol in combination with one or more other ingredients such as a surface adherent, thickener, emulsifier, preservative, breath freshener, antimicrobial, and a sweetener. Artificial color is optionally added. In certain embodiments, the oral hygiene compositions comprise xylitol together with a preservative and a breath freshener (e.g., a sorbate) and an antimicrobial (e.g., a benzoate). In certain other embodiments, the oral hygiene compositions comprise xylitol together with a preservative (e.g., a sorbate, a benzoate, chlorhexidine, chlorhexidine gluconate, chlorhexidine digluconate) and a breath freshener (e.g., a gluconate such as zinc gluconate, a sorbate such as potassium sorbate) and an antimicrobial (e.g., a benzoate, a chlorhexidine).

In another embodiment, the compositions of the present invention contain xylitol and an enzyme, emilgase, in combination with one or more other ingredients such as a surface adherent, thickener, emulsifier, preservative, breath freshener, antimicrobial, and a sweetener.

In one embodiment the compositions of the invention further comprise antimicrobial and/or anti-plaque agents. In an alternative embodiment, the compositions of the present invention further comprise antibiotics, antifungals, antiseptics, immunomodulators and/or anticancer drugs. In yet another embodiment, the compositions of the invention further comprise taste-enhancing components that may be selected according to the user's preference.

In a specific embodiment, an oral hygiene composition of the present invention, also known as BREATHALYSER® (imRex Inc., Ontario, Canada), comprises a combination of xylitol (a natural sweetener found widely distributed in nature for example in strawberries, plums, pears, hardwoods such as birch trees, mushrooms, lettuce, and corn cobs) with other naturally-occurring nutrients and food preservatives that are generally recognized as safe and as having antimicrobial and tartar reduction activity. These ingredients are optionally combined with an antibacterial preservative, such as chlorhexidine, in the correct proportions to provide a beneficial effect. Chlorhexidine is a preferred additive and may be used in different forms such as gluconate, dihydrochloride, acetate and diacetate salts or as pure chlorhexidine.

In an additional embodiment, the compositions of the present invention further comprise emilgase. Emilgase is an enzyme and is commercially available in a finished, spray-dried form from Rocky Mountain Natural Laboratories, Boulder Colo., USA.

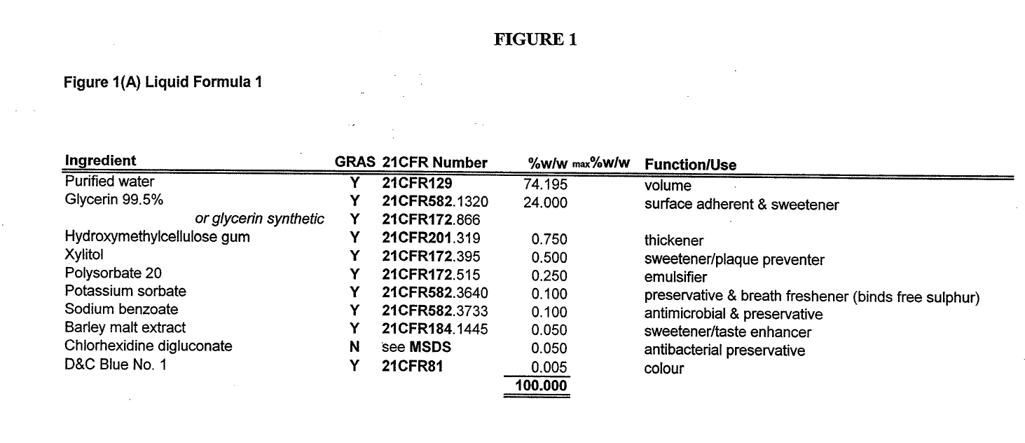

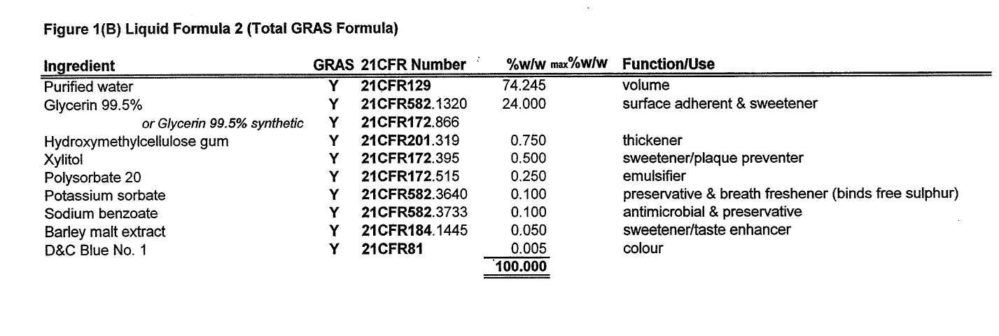

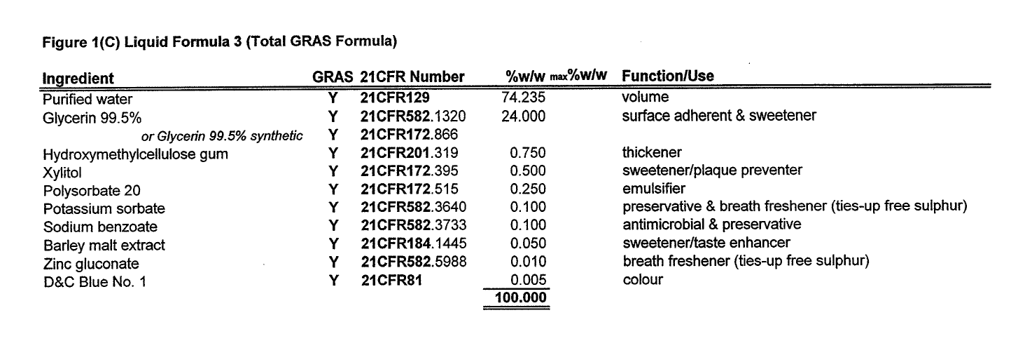

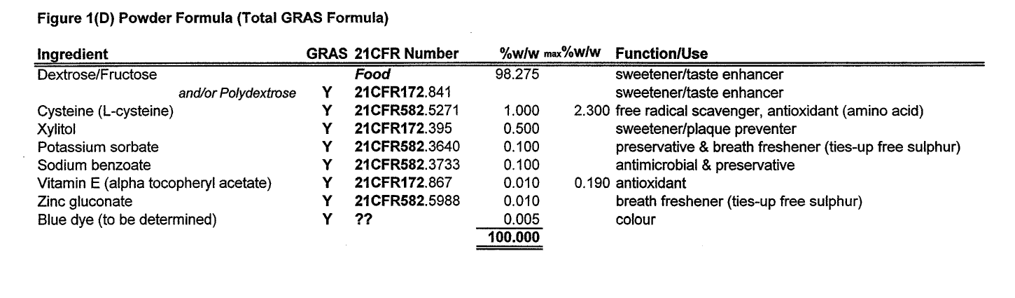

The principal ingredients of one embodiment of the oral hygiene composition (together with their main function/use) of the present invention (also known as BREATHALYSER® (imRex Inc., Ontario, Canada)), are listed below in Table 1. FIG. 1 provides specific formulations for various embodiments (including, but not limited to liquid formulas, powder formulas, sachets, tablets, capsules, dissolving capsules, and effervescent powder formulas).

TABLE 1
Oral Hygiene Composition

| Ingredient | Function/Use |
|---|---|
| Purified water | Volume |
| Glycerin | Surface adherent and sweetener |
| Hydroxymethylcellulose gum | Thickener |
| Xylitol | Sweetener/plaque preventative |
| Polysorbate 20 | Emulsifier |
| Potassium sorbate | Preservative & breath freshener |
| | (binds free sulphur) |
| Sodium benzoate | Antimicrobial & preservative |
| Barley malt extract | Sweetener/taste enhancer |
| Chlorhexidine digluconate | Antibacterial preservative |
| D&C Blue No. 1 | Color |
| Zinc gluconate | Immune stimulant and breath freshener |
| | (binds free sulphur) |
| Dextrose/fructose/polydextrose | Sweetener/taste enhancer |
| Cysteine | Free radical scavenger, antioxidant |
| Vitamin E | Antioxidant |
| Sodium bicarbonate | CO₂ source |
| Malic acid | For reacting with sodium bicarbonate |

Emilgase is produced by the co-culture of two non-pathogenic, mesophilic species of bacteria from the genus Bacillus (Bergey's Manual of Systematic Bacteriology, (1986) 8^{th} Ed. Vol. 2: the family Bacillaceae features six genera of Gram's positive, endospore-forming bacteria. Bacillus spp. bacteria are distinguished from the other endospore-forming bacteria on the basis of being strict or facultative aerobes, rod-shaped, and (usually) catalas-positive).

The general outlines for production and purification of the Bacillus spp. enzymes are outlined in U.S. Pat. No. 4,331,762 and discussed in the Examples below.

In one embodiment, the oral hygiene compositions of the present invention optionally comprise emilgase in varying amounts. This oral hygiene composition, including the emilgase, is also known as BREATHALYSER® (+) (imRex Inc., Ontario, Canada). In certain embodiments, emilgase is added to test products and systems at a rate of 0.05% w/w of the basic oral hygiene composition. This addition may range from 0.001% to 50% w/w, or 0.001% to 10% w/w. One preferred addition is approximately 0.05% however other suitable ranges include 0.005% to 5.0%, 0.01% to 2.5% and 0.02% to 2.0%

Unlike most pet oral hygiene products that use sodium chloride or stabilized chlorine dioxide as their base, the present invention does not incorporate such ingredients. Although sodium chloride and stabilized chlorine dioxides exhibit strong antimicrobial activity, a number of opinions from veterinary dental experts suggest that prolonged use of these chemicals may cause damage to the oral mucosa itself. Some go further, and say that after prolonged use they simply cease to be as effective.

Currently available oral hygiene products for pets do not have the key active ingredients found in the present invention. Many have surfactants that actually change the saliva from an acidic to an alkaline pH. This appears to be the main mechanism of action for most of these products.

Additional ingredients that are known to kill microorganisms, reduce inflammation and rebuild damaged tissue may also be included in the compositions of the present invention: oleuropein, pimaricin (natamycin), nisisn, xanthorrhizal, lactoferrin, colloidal silver, aloe vera, folic acid, calendula flower, echinacea pupurea, gota kola extract, chlorophyll, phytoplenolin extract, chamomile flower, blood root, prickly ash bark, green tea leaf, oregano leaf, peppermint oil, cinnamon bark, bee propalis, eucalyptus leaf, lavender oil, bio-saponin, co-enzyme Q-10, olive leaf extract, black walnut green hulls, clove leaf, thyme herb, grapefruit seed extract (citricidal), and vegetable glycerin. As will be appreciated by those skilled in the art, substitutions may be made with similar or equivalent ingredients. All such substitutions and replacements are considered to be within the scope of the present invention.

In addition to the ingredients listed above and in Table 1, the following ingredients may also be incorporated into the compositions of the present invention: lactic acid (pH adjuster), citric acid (pH adjuster), sorbitol (sweetener), hyaluronic acid (mucosal membrane protectant), potassium thiocyanate (preservative) and methyl salicylate (taste enhancer). As will be appreciated by those skilled in the art, substitutions may be made with similar or equivalent ingredients. All such substitutions and replacements are considered to be within the scope of the present invention.

As used herein, the phrase “oral hygiene composition” is intended to encompass the various embodiments of the compositions described herein, together with substitutions and equivalents as would be appreciated by those skilled in the art. In addition, the phrase “oral hygiene composition” is intended to encompass compositions that are useful for all aspects of oral hygiene, including but not limited to, preventing oral disease, maintaining oral health, decreasing or eliminating bad breath, whitening teeth, preventing gum deterioration, and preventing tooth decay.

### Forms for Administration of the Compositions of the Present Invention

The novel oral hygiene compositions of the present invention may be placed in a variety of forms for administration to animals or humans. For example, the compositions may take the form of a liquid, spray, paste, gel, foam, syrup or powder. Additionally, the compositions may be incorporated into toothpaste, lozenges, lollipops, tablets, flash melt formulations, gums, candy or beverages. Other applications of the compositions include use as a coating on a strip to be applied to teeth, use as coating on dental floss, as an additive in solution introduced into the oral cavity, as a gel or paste to be applied to dental molds (such as molds used for whitening teeth) or for orthodontic applications. As would be recognized those skilled in the art, certain embodiments are more suitable for human use (toothpastes, mouthwashes, lollipops, teething toys) whereas others are more suitable for animal use (dental chew and toys (i.e. rawhide chews), biscuits, cookies, treats).

As demonstrated in the Examples below, the embodiment of oral hygiene composition of the present invention is not damaged by baking at temperatures higher than approximately 55° C. for approximately 16-18 hours and at approximately 180° C. for 45 minutes and therefore could be incorporated into foods for animals or humans. Animal foods include, but are not limited to, dry kibbles, cubes and canned foods, treats, cookies, sticks and chews. Chews are generally manufactured from beef hides, but could be produced from pig, sheep, goat, poultry and fish skin products. The production outline for beef rawhide chews comprising compositions of the present invention is presented below in the Examples.

Additionally, just as the compositions of the present invention may be incorporated into animal foods such as treats, they may also be incorporated into human foods such as cookies, crackers and other foods. Such an embodiment would be particularly suitable for items such as teething biscuits for babies and young children. In addition, the compositions of the present invention may be incorporated into beverages including but not limited to, carbonated beverages, bottled waters, flavored waters, sports drinks, juices and nutritional supplements.

The compositions of the present invention may comprise a gel type format comprising, purified water, glycerin 99.5%, hydroxymethylcellulose gum, polysorbate 80, barley malt syrup chlorhexidine digluconate, zinc gluconate, and/or coloring. One specific embodiment of such a composition is provided below in Example 8.

### Methods for Using the Compositions of the Present Invention

The present invention is useful for animals including but not limited to farm animals and zoo animals. For example, the compositions of the present invention may easily be incorporated into drinking water administered to amphibians: frogs, salamanders, reptiles: snakes, lizards, crocodiles, turtles, aves: penguins, ostriches, parrots, mammals, marsupials: kangaroos, koala bears; hoofed animals: sheep, pigs, giraffe, zebra; carnivores: bears, otters, weasels, marine animals: dolphins, porpoises; primates: monkeys, orangutans, gorillas, chimpanzees; and felines: lions, tigers, panthers, leopards.

The present invention is especially useful for domesticated animals, including but not limited dogs, cats, rabbits, guinea pigs, hamsters, gerbils, ferrets and horses. Because of the unique properties of the present invention, even picky animals such as finicky cats will accept the novel compositions in their drinking water. The compositions may comprise unique formulas that have a pleasant, sweet taste that has been carefully developed and tested to provide a very palatable solution for any animals, including dogs and cats, and to ensure their long term, daily acceptance of the product.

The present invention comprises an effective product optionally containing proven antimicrobial and anti-plaque agents. The ease-of-use of the compositions supports the veterinarian's goal of improving pet dental care between clinic examinations. In one embodiment when the present invention is used daily as directed, it provides an effective oral care option for most pet owners.

The benefits of using the novel oral hygiene compositions of the present invention become readily apparent after just a few uses. Reports indicate that early results are often noticeable, even after just two weeks of use. During the studies discussed herein, after using just a preliminary amount of the novel oral hygiene compositions described in the Examples and Figures herein, respondents for both dogs and cats scored the overall improvement level or reduction of the pet's existing problem as follows: 93% reduction in halitosis and 85% reduction in tartar build-up.

An important property of the present invention is that it acts to counteract the causes of oral hygiene problems. For example, though not wishing to be bound by the following theory, the key ingredients react with and bind free sulfur, a major contributor to foul-smelling breath. Other ingredients destroy bacteria that form the sticky plaque on teeth and at the tooth-gum junction. By attacking the major causes of bad breath, and improving the overall hygiene of the oral cavity, the present invention helps control halitosis. It does not just cover an objectionable odor with a more acceptable one.

The present invention is further desirable because it reduces gingivitis. Gingivitis, especially at the gum line, results primarily from persistent microbial growth in the oral cavity. By reducing existing bacterial levels and the associated tartar build-up, gum inflammation can be reduced by use of the novel compositions described herein.

It should be understood that the foregoing relates only to preferred embodiments of the present invention, and that numerous modifications or alterations may be made therein without departing from the spirit and the scope of the invention as set forth in the appended claims. This invention is further illustrated by the following examples, which are not to be construed in any way as imposing limitations upon the scope thereof. The references cited herein are hereby incorporated by reference in their entireties, including U.S. Provisional application Ser. No. 60/532,303.

### Example 1

### Concentrated Drinking Water Additive

The present example describes a formulation for a composition useful for maintaining oral hygiene in animals. Unlike direct application products, this formulation may be used as a concentrated drinking water additive. It is a pleasant tasting, alcohol-free solution that has been formulated to help freshen an animal's oral cavity, decrease bad breath and maintain oral hygiene.

The drinking water is prepared by adding 10 mL (two capfuls) of the formulation (presented below in Table 2) per one quart of fresh drinking water. In certain embodiments, the formulation comprises 10 mL per one liter (33.8 US fluid ounces (fl. oz.)) of fresh drinking water. It is contemplated that most animals do not need to consume more than 10 mL of the formulation per 100 kg of body weight per day. All animals will require additional fresh drinking water if the treated water is fully consumed. It is recommended, but not required, that the treated drinking water be prepared fresh each day.

TABLE 2
Oral Hygiene Composition

| Ingredient | % w/w | Range | Function/Use |
|---|---|---|---|
| Purified water | 74.195 | q.s. | volume |
| Glycerin 99.5% | 24.0 | ±10% | surface adherent |
| Hydroxymethylcellulose | 0.75 | ±10% | thickener |
| gum | | | |
| Xylitol | 0.50 | ±10% | sweetener & plaque |
| | | | preventative |
| Polysorbate 20 | 0.25 | ±10% | emulsifier |
| Potassium sorbate | 0.10 | ±10% | preservative & |
| | | | breath freshener |
| | | | (binds free sulphur) |
| Sodium benzoate | 0.10 | ±10% | antimicrobial & |
| | | | preservative |
| Barley malt extract | 0.05 | ±10% | sweetener & taste |
| | | | enhancer |
| Chlorhexidine digluconate | 0.05 | ±10% | antibacterial |
| | | | preservative |
| D&C blue #1 | 0.005 | ±10% | Color |
| | 100.00 | | |

### Example 2

### Effect of Oral Hygiene Composition on Halitosis, Tartar Build-Up and Gum Health

The oral hygiene composition of the present invention comprising the formulation, set forth in Table 2 above, was provided to 74 animals. Subsequently, data concerning halitosis, tartar build-up and gum health were collected from professional caregivers. The results, shown below in Table 3, demonstrate that the novel composition of the present invention is effective in reducing halitosis and tartar build-up and in improving gum health.

### Design and Data Collection

The data were obtained from 21 professional caregivers (veterinarians, veterinary technicians, groomers and kennel personnel) who were associated with animals where the owners had concerns regarding bad breath (halitosis). All animals were treated with the novel oral hygiene composition of the present invention for at least eight weeks and then re-evaluated by the original prescriber.

Each caregiver was visited by the same data-collector. Responses to specific questions were graded on a five-point verbal analogue scale. A final, ‘yes/no,’ global question was then asked regarding the continued use and future prescribing of the product by each caregiver.

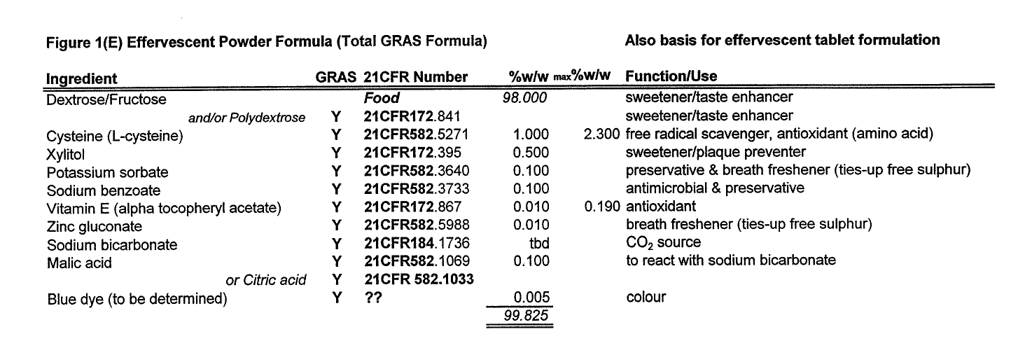

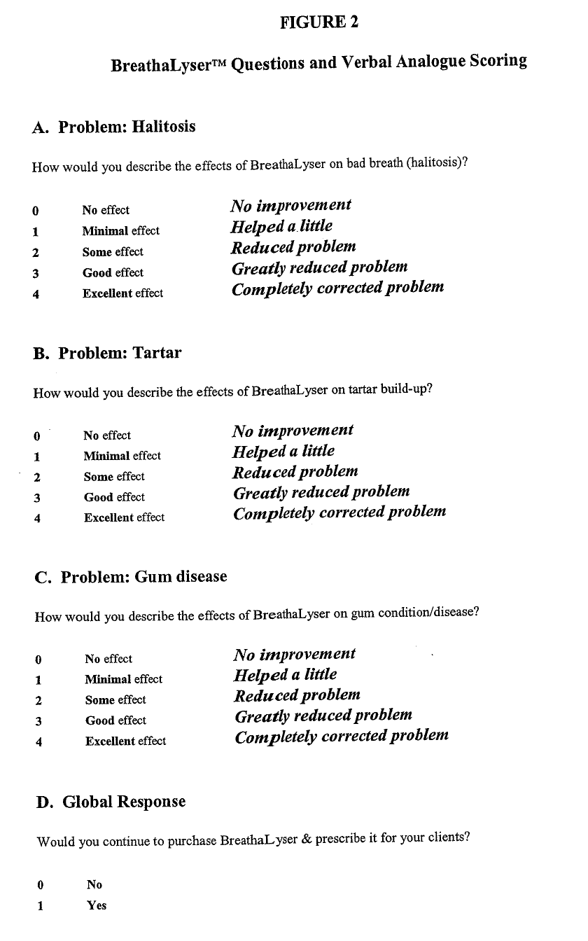

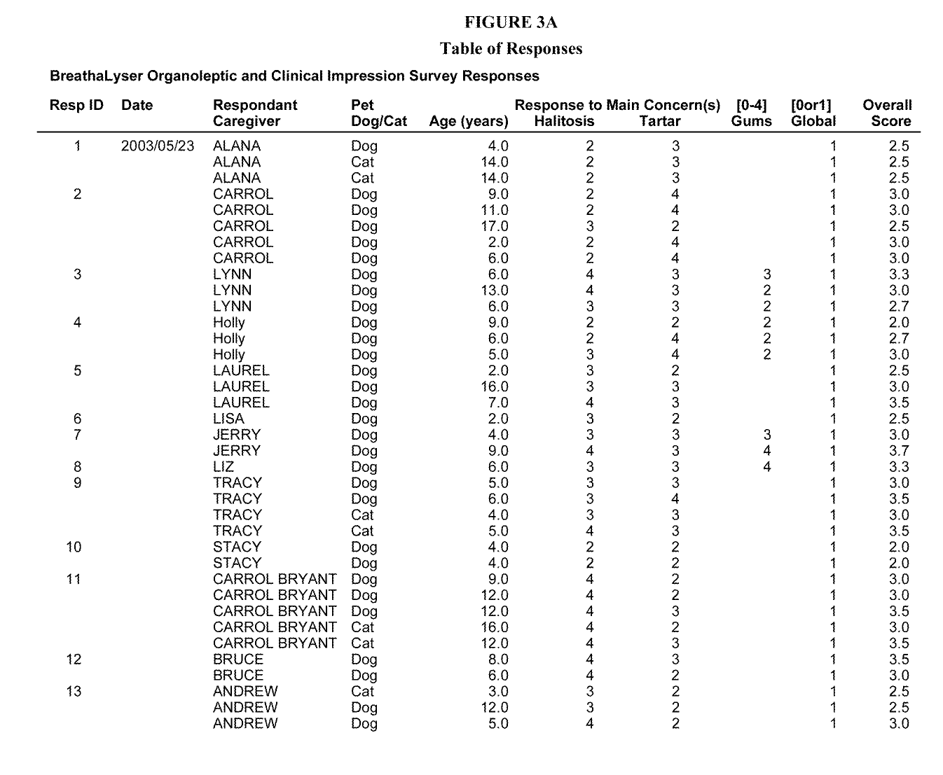

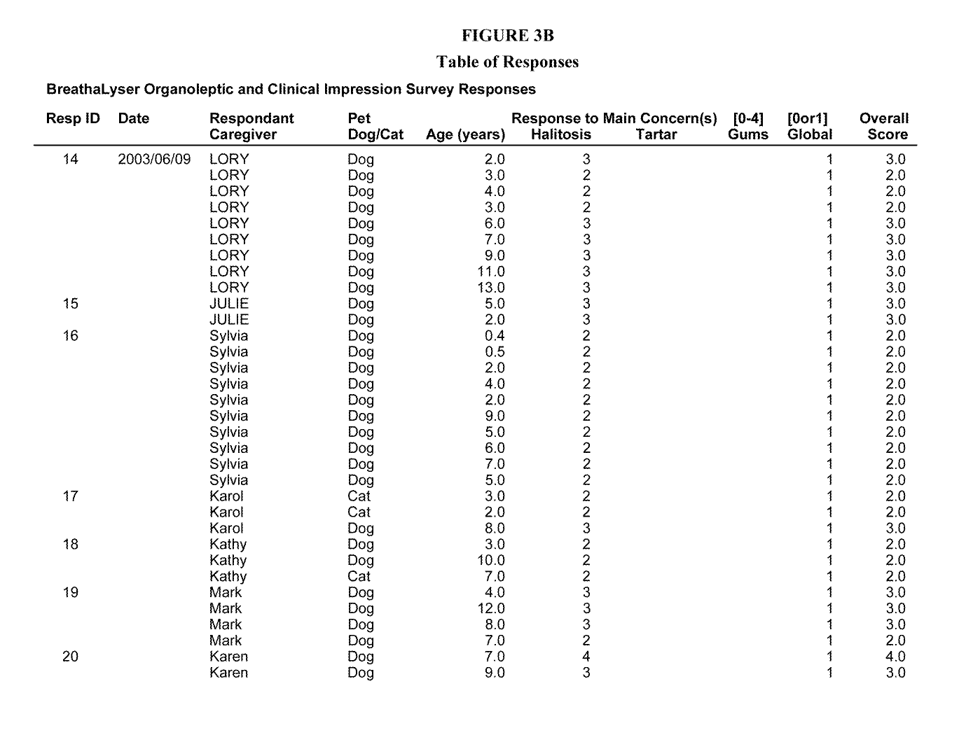

Responses on 74 animals (62 dogs and 12 cats) were obtained for the reduction of halitosis. Forty-two responses were related to reduced tartar formation (30 dogs and 12 cats). Gum health was a concern in nine dogs. FIG. 2 provides questions and verbal analogue scoring used in the study and FIG. 3 provides a table of responses accompanying the study.

### Statistical Analysis

The means for each question were evaluated by a chi-square analysis comparing the values to a base score of zero (no effect). A maximum mean score of 4.0 was used for the residuals for each analysis.

TABLE 3

| | Age | | | Halitosis | | | | Tartar | | | | Gum Health | | | | Global | |
|---|---|---|---|---|---|---|---|---|---|---|---|---|---|---|---|---|---|
| | mean | | | mean | | | | mean | | | | mean | | | | | Buy/ |
| Animal | years | SD | n | score | SE | n | p≦ | score | SE | n | p≦ | score | SE | n | p≦ | n | Reuse |
| Dogs | 6.6 | 3.7 | 62 | 2.790 | 0.095 | 62 | 0.05 | 2.867 | 0.142 | 30 | 0.05 | 2.667 | 0.289 | 9 | 0.05 | 21 | 21/21 |
| Cats | 9.7 | 5.0 | 12 | 2.917 | 0.260 | 12 | 0.05 | 2.667 | 0.142 | 12 | 0.05 | N/A | | | | 8 | 8/8 |
| All Animals | 6.9 | 4.0 | 74 | 2.811 | 0.090 | 74 | 0.05 | 2.810 | 0.109 | 42 | 0.05 | N/A | | | | 21 | 21/21 |

### Discussion

Overall, there was a statistically significant response (p<0.05) for all three ascertained problems. However, the power of detection (1-0 was greater than 0.90 for only halitosis and reduced tartar build-up.

It should be noted that the overall (global) response for future purchase and use of the product, by the interviewed caregivers, was 100% (21/21).

### Example 3

### Rawhide Chews Comprising Oral Hygiene Compositions to Reduce the Reoccurrence of Halitosis, Plaque, Calculus and Gingivitis in Dogs Following Routine Dental Cleaning

The present study comprises a 42-day, randomized, controlled, proof of principal study to clinically evaluate the use of rawhide chews impregnated with oral hygiene compositions in dogs (impregnated chews). The chews were 1.5 g (approx.) beef rawhide chews soak-impregnated with 5 mL of the oral hygiene composition described in Table 2 and dried by low temperature baking. Four dogs were provided with one impregnated rawhide chew per day for 42 days and two dogs were given no rawhides (neither natural nor impregnated). The dogs used in the study comprised mature, medium sized dogs with halitosis and tarter sufficient to require dental cleaning.

### Protocol

Each animal evaluated as a possible candidate for the study was only enrolled with the informed consent of the owner and the owner's commitment to comply with the treatment regimen and to return the animal for re-evaluation at 42 days after the initial dental procedure (Owner's Informed Consent and signature). Animals evaluated and selected by the principal investigator and entered into the trial had the information and evaluation results entered into the tb (baseline) area of the Clinical Report form. Test and control animals were randomized from a table of random numbers. The designation of each animal was by order of entry into the trial and was contained in sealed, numbered envelopes. Each animal was anesthetized by normal procedures for this practice.

As used herein, tb and t0 were the same day: tb was immediately after anesthesia and before dental cleaning; to was immediately after dental cleaning and before the animal recovered from anesthesia.

Baseline (tb) digital photographs of the left and right dental arcades were taken and appropriately labeled or identified for future reference. Teeth were cleaned (and polished if required) to remove scale and calculus and any necessary gum procedures were performed as is normal for this practice. At the beginning of the study (t0) digital photographs of the left and right dental arcades were taken and appropriately labeled or identified for future reference. Any comments or concerns regarding the procedure or study outcome were recorded in the t0 area of the Clinical Report form.

Treatment group owners were prescribed 42 impregnated chews and instructed to feed their animal one chew per day. If the test subject did not want to chew the rawhide due to mouth irritation on any day after the dental procedure at t0, the rawhide was removed and discarded and a new rawhide offered the following day. Owners returned the test animal for oral examination at 42 days (t42), the day the last rawhide test product was administered. The principal investigator re-evaluated the animal and entered the evaluation results in the t42 area of the Clinical Report form. Day t42 digital photographs of the left and right dental arcades were taken and appropriately labeled or identified for future reference. The principal investigator recorded any comments or concerns regarding the efficacy or treatment. Owner Rawhide Rejection Logs were collected and any owner comments or concerns were recorded in the t42 area of the Clinical Report form. Owners were asked if they would continue to purchase and use this product if it was commercially available and the response was recorded in the t42 area of the Clinical Report form. Results are presented in Table 4.

TABLE 4

| | | Mean | Mean | Improvement from Baseline at 42 days | | | | |
|---|---|---|---|---|---|---|---|---|
| | | Age | Wt | | | | | Total |
| Group | No. | (years) | (kg) | Halitosis | Plaque | Calculus | Gingivitis | Score |
| Control | 2 | 6.9 | 34.7 | 1.00 | 0.50 | 0.00 | 0.00 | 1.50 |
| Treated | 4 | 4.9 | 23.5 | 2.25 | 2.00 | 1.38 | 1.75 | 7.38 |
| | | | anova | | | | | p < 0.01 |

All animals accepted the treats, however, one toy poodle (6.9 years old and 4.1 kg wt) had problems consuming the entire treat in one day. All owners indicated that they would use the product if commercially available.

### Example 4

### Methods for Manufacturing Rawhide Chews

Two lab scale trials were conducted in order to determine effective methods for manufacturing rawhide chews.

### Trial #1

In trial #1 (formulation in Table 5), the beef rawhide and pork ears were soaked in the oral hygiene solution described above in Table 2 with added color (to color the hides brown for appeal), for 2 hours, removed from the solution, placed on trays and then dried overnight (18 hours) in a constant temperature incubator at 55° C. Four samples were weighed initially, after soaking and then after drying. The weights are shown below in Table 6.

TABLE 5
Formulation for Trial 1

| Ingredient | % | g batch |
|---|---|---|
| Water | 1.00 | 20.000 |
| Oral Hygiene solution (see Table 2) | 98.50 | 1970.000 |
| Caramel color | 0.50 | 10.000 |
| Total | 100.00 | 2000.000 |

TABLE 6
Weight measurements and % solution uptake for
Trial 1 beef rawhide and pork ear samples

| | | | % Solution | Final Wt. After |
|---|---|---|---|---|
| | Initial Wt. | Soaked Wt. | Uptake | Drying |
| Sample # | (g) | (g) | (w/w basis) | (g) |
| Beef 1 | 10.9 | 16.9 | 55 | 10.9 |
| Beef 2 | 15.7 | 23.1 | 47 | 15.4 |
| Beef 3 | 20.0 | 33.2 | 66 | 20.3 |
| Beef 4 | 43.8 | 58.0 | 32 | 43.9 |
| Pig 1 | 13.6 | 17.5 | 29 | 13.9 |
| Pig 2 | 12.0 | 15.3 | 27 | 12.3 |
| Pig 3 | 12.9 | 15.5 | 20 | 13.2 |
| Pig 4 | 10.0 | 12.8 | 28 | 10.4 |

### Discussion

The solution uptake was acceptable for the beef rawhide but less acceptable for the pig ears. The fat content of the pig ears is higher than the rawhide (27% vs. 0.6%), which can prevent solution uptake. The % solution uptake varied among the samples. This is a result of the varied sizes and thickness of each piece. For example, the thicker the piece, the lower the % solution uptake based on a weight per weight basis. The appearance of the rawhide was very light brown, and as would be recognized by those skilled in the art, the color can be adjusted according to the preference of the intended user. The drying time was determined by the time required to dry the hides to a constant weight (original weight). However, due to the varied size and thickness, some pieces may have a lower weight than the original weight, and as a result, a lower final moisture content.

### Trial #2

In trial 2 (formulation in Table 7), a similar process was used for soaking and drying the rawhide and pig ears. The color was increased in the soak solution to darken the rawhide color. After the rawhide was soaked and dried, the enzyme, emilgase, was sprayed onto one side of the rawhides and pig ears, then dried again for two hours at 50° C. (lower temperature to protect the enzyme). Emilgase spray attempted to achieve a coating of 3.5 g of the enzyme per 1 kg of finished product. Spray coating was 3500 mg in approximately 100 mL of oral hygiene solution (˜3.5% solution)

For commercial production with emilgase, the product was cooled to less than 50° C. Next, the emilgase solution was sprayed or painted on the upper surface in an amount appropriate for achieving approximately 2.5 to 5 mg emilgase per one days ration of finished product. The product was dried at 50° C. for at least 2 hours. The weight measurements for trial 2 can be found in Table 8 below.

TABLE 7

| Ingredient | % | g batch |
|---|---|---|
| Water | 0.00 | 0.000 |
| Oral hygiene solution (see Table 2) | 98.50 | 1970.000 |
| Caramel color | 1.50 | 30.000 |
| Total | 100.00 | 2000.000 |

TABLE 8

| | | | % Solution | Final Wt. After |
|---|---|---|---|---|
| | Initial Wt. | Soaked Wt. | Uptake | Drying |
| Sample # | (g) | (g) | (w/w basis) | (g) |
| Beef 1 | 15.8 | 20.8 | 32 | 15.4 |
| Beef 2 | 9.1 | 14.8 | 63 | 9.1 |
| Beef 3 | 36.3 | 44.8 | 23 | 35.3 |
| Beef 4 | 30.7 | 37.0 | 21 | 29.7 |
| Pig 1 | 9.1 | 11.5 | 26 | 9.2 |
| Pig 2 | 21.3 | 25.5 | 20 | 21.6 |
| Pig 3 | 10.2 | 12.2 | 20 | 10.3 |
| Pig 4 | 15.3 | 19.1 | 25 | 15.7 |

### Discussion

The color was darker than in trial 1. Similar lower % solution uptake results were found with the pig ears.

### Pilot Scale Trial

Due to the lower % solution uptake of the pig ears, beef rawhide was selected for scale up. About 15 kg of rawhide was soaked and dried based on the process developed in the lab scale trials (composition in Table 9). The rawhide was soaked in solution for 2 hours, then removed from solution and placed on trays. The rawhide was then dried in constant temperature at 55° C. for 16.5 hours. The enzyme, emilgase (diluted in oral hygiene solution) was sprayed on one side of the rawhide and pig ears, then dried again for 2.5 hours at 50° C. (lower temperature to protect the enzyme). Emilgase spray attempted to achieve a coating of 3.5 g of the enzyme per 1 kg of finished product. Spray coating was 3500 mg per approximately 100 mL oral hygiene solution (˜3.5% solution). The weight measurements of the samples of rawhide are shown in Table 10.

TABLE 9

| Ingredient | % | g batch |
|---|---|---|
| Water | 0.00 | 0.000 |
| Breathalyser solution | 98.50 | 137.900 |
| Caramel color | 1.50 | 2.100 |
| Total | 100.00 | 140.000 |

TABLE 10
Weight measurements and % solution uptake
for pilot scale trial beef rawhide samples.

| | | | % Solution | Final Wt. After |
|---|---|---|---|---|
| | Initial Wt. | Soaked Wt. | Uptake | Drying |
| Sample # | (g) | (g) | (w/w basis) | (g) |
| Beef 1 | 22.5 | 28.7 | 28 | 21.1 |
| Beef 2 | 18.6 | 23.2 | 25 | 17.8 |
| Beef 3 | 28.7 | 36.1 | 26 | 27.3 |
| Beef 4 | 17.6 | 22.4 | 27 | 16.3 |
| Beef 5 | 21.4 | 27.8 | 30 | 20.7 |

### Discussion

The process for drying was modified to decrease the drying time from 18 hours to 16.5 hours for the first drying stage and from 2 hours to 2.5 hours for the second drying stage. The pilot-scale drying oven had improved circulation of air and therefore the initial drying stage was shortened. The second drying stage was extended slightly to account for the time required to bring the oven to the correct temperature after the door is closed to the oven. Drying time is dependent on equipment capability such as size, air circulation, humidity control, etc. In conducting the experiments described above, Caramel Color was obtained from Sensient Colors Canada Ltd. (Kingston, Ontario); Beef Rawhide Chips and Pig Ears were obtained from Euro-Can Pet Products (New Hamburg, Ontario).

### Example 5

### Production of Emilgase

In one embodiment of the oral hygiene composition of the present invention, the composition further comprises the enzyme called emilgase. This oral hygiene composition, including the emilgase, is also known as BREATHALYSER® (+) (imRex Inc., Ontario, Canada). The following describes general procedures for the production of emilgase. Emilgase is available from Benjamin Fuchs, at Rocky Mountain Natural Laboratories, Boulder, Colo., USA.

### Culture

The bacillus bacteria are cultured aerobically in commercial Soy-Bean Casein Digest Media (BD Bacto™ 211823) without additional ingredients at 37 to 42° C. Culture continues until the later stage of the logarithmic growth phase and prior to spore formation. The appropriate growth stage can be determined by optical density.

Additional components may be added to the media as determined by one skilled in the art; sucrose as a carbon and energy source, K₂HPO₄ or KH₂PO₄ as a buffer and source of potassium and sodium, (NH₄)₂HPO₄ as a buffer and source of nitrogen and phosphorus, MgSO₄.7H₂O as a source of magnesium, FeSO₄.7H₂O as a source of iron, MnSO₄.7H₂O as a source of manganese.

### Enzyme Purification

In general, commercially useful enzymes can be isolated and purified from a bacillus culture by centrifugation with a De Laval, or equivalent type, centrifuge. The collected cells are then subjected to conventional enzyme isolation and purification techniques: the cells are disrupted and centrifuged to provide a supernatant that is either fractionated with an organic solvent or a variety of salts such as sodium chloride, magnesium sulfate, ammonium sulfate, sodium sulfate, potassium phosphate, sodium citrate, or purified by adsorption onto a carrier.

Emilgase can be purified by techniques described above, and the physicochemical properties of the resulting product can be compared with those of a reference sample retained from an earlier extracted enzyme preparation. A single band being produced toward the positive electrode with an acrylamide gel electrophoresis or SDS electrophoresis technique indicates the purity of the enzyme.

Emilgase appears to be a construct of enzymes produced by interaction during growth of the individual bacillus bacteria. Electrophoresis results demonstrate a different emilgase band to any of the other enzyme bands produced by either of the individual bacteria.

### Enzyme Drying

Emilgase can be spray dried with a Filtermat Spray Dryer, Type FMD-12.5 (Gea Niro Inc., Columbia, Md.) or equivalent, to produce a non-dusty, agglomerated, easy-to-use powder that can be stored in airtight containers at room temperature.

### Formulations

To date, emilgase has been added to all test products and systems at a rate of 0.05% w/w of the basic oral hygiene composition. This addition could range from 0.01% to 2.5%. One preferred addition is 0.05% although the following ranges are also envisioned: 0.001% to 10.0%, 0.005% to 5.0%, 0.01% to 2.5%, and 0.02% to 2.0%

### Example 6

### Oral Hygiene Composition with Emilgase for the Treatment of Cats with Recalcitrant Stomatitis

Several cats experiencing recalcitrant stomatitis were treated using oral hygiene composition of the present invention further comprising emilgase. All compositions comprised 0.05% emilgase. All treatments were 5 mL/500 mL water (10 mL/L rate). If additional water was required after the 500 mL was consumed, plain water was offered. Any unconsumed solution was discarded after 24 hours and fresh solution was mixed.

### Cat #1

Cat #1 (13 year old, domestic short haired, neutered male) suffered from oral stomatitis for 11 years. Over the last 11 years, Cat #1 had been treated with Metronidazole, Clindamycin, Depo-Medrol injections, and total teeth extractions. Biopsy of the stomatitis revealed suppurative exudative inflammation, and no etiologic agent was found. Despite multiple drug prescriptions and removal of all teeth, Cat #1 suffered stomatitic lesions in the back of mouth every 4 to 6 weeks to such a degree that Cat #1 could not eat. Cat #1 was administered the oral hygiene composition of the present invention with emilgase, 1 capful daily in water. Within 2 weeks all lesions cleared up and no further medications were needed.

### Cat #2

Cat #2 (13 year old, domestic short haired, spayed female) suffered from severe stomatitis since October 1999. She was treated for extended periods with Antirobe liquid. All of Cat #2's teeth were extracted in December 1999 and a biopsy of oral mucosa revealed severe lympho-plasmocytic gingivitis. Treatment since 1999 has been bimonthly injections of Depo-Medrol. Cat #2 was administered the oral hygiene composition of the present invention with emilgase. Within 2 weeks of administration and continuous use, all lesions cleared and to date no further medication was necessary.

### Cat #3

Cat #3 (seven year old, domestic short haired male) had suffered from stomatitis for 6 months. During this time he tested positive for feline immunodeficiency virus. He was treated for 3 months with antibiotics and pain medications, including 20 mg/kg of Zithromax every 48 hours for 10 days, 10 mg/kg of Antirobe every 12 hours, and injectable Tolfedine 20 mg every 48 hour. The cat deteriorated further and displayed stomatitis, gingivitis, periodontal disease, plaque formation, etc. Cat #3 was treated with the oral hygiene composition of the present invention with emilgase at 1 capful per 500 mL of drinking water. After approximately 5 days of treatment, Cat #3 exhibited signs of improvement and no longer required antibiotics or pain medication. Most importantly, no evidence of anorexia was present and the cat's oral condition improved dramatically.

### Cat #4

Cat #4 (four year old domestic short haired male) experienced weight loss, severe anemia and parasite infestation as a kitten. He was treated with Antirobe and Zeniquin for recurrent stomatitis. Although he responded well to Zeniquin for 10 days, he relapsed, and again displayed stomatitis. The caregiver resorted to the oral hygiene composition with emilgase alone. Shortly thereafter (approximately a few months), Cat #4 demonstrated a response to the product with little evidence of stomatitis and no anorexia.

### Cat #5

Cat #5 (five year old domestic short haired male) had a history of weight loss for 2 years and suffered from recurrent stomatitis that required extensive dental work. Treatment with Zithromax 80 mg caused temporary responses, with repeated occurrences of stomatitis. Following treatment with the oral hygiene composition with emilgase over a period of approximately six months, it became evident that oral hygiene composition alone had controlled the condition and Cat #5 continued to feel more comfortable, experience less pain and maintain a good appetite.

### Cat #6

Cat #6 (domestic long hair, mature) presented with acute stomatitis and was treated with Zithromax. Cat #6 did not respond to the antibiotic. For approximately six months, the operator fed the cat oral hygiene composition with emilgase continually in the drinking water. Cat #6 responded very well and the stomatitis was resolved.

### Example 7

### Tooth Whitening/Brightening After 42 Days Treatment with Rawhide Chews Impregnated with Oral Hygiene Composition Comprising Emilgase

Four to six photographs of the teeth on each side of the mouth were taken with the same digital camera and lighting conditions before any dental procedures, at baseline (tb), immediately after dental cleaning on day 0 (t0) and on re-evaluation at day 42 (d42).

### Procedure

The protocols for this example are the same as those described in Example 3. The rawhides used for this study were impregnated with the oral hygiene compositions of the present invention further comprising emilgase.

One image from each side of each animal's mouth was chosen for times t0 and t42 on the basis of equivalent gum color (as representative of the teeth color for whiteness evaluation). Evaluation of the whiteness shade for left and right side upper canine and upper first molar teeth was performed by comparison to an Arm & Hammer Whitening Shade Guide scale (Church & Dwight, Mississauga, Canada) where the first and twelfth shades were dropped and the remaining shades (2-11) were numbered 1-10.

All images were displayed on the same computer screen under the same lighting conditions and the whiteness shade was selected by comparison of the scale to the evaluation tooth.

A positive response for each tooth was indicated by a reduction in the scale number from t0 to t42 and by subtracting the t42 score from the t0 score. Overall color change for all four evaluated teeth was represented by the sum of the t0-t42 scores for each animal. The mean change for each group (treated and control) was reported.

### Results

At 42 days, the mean change in whiteness/brightness for the treated group was 1.5 compared to −1.0 for the control group. This difference was not statistically significant by ANOVA evaluation. The number of animals responding positively was not statistically significant by Chi-square at 95% confidence (α=0.05). However, it was significant at 90% confidence (α=0.1).

### Discussion and Conclusion

This study demonstrates that the oral hygiene composition comprising emilgase, when administered over 42 days, will help maintain the whiteness and brightness of a dog's teeth and may improve the overall whiteness of the teeth.

### Example 8

### Oral Hygiene Composition Gum Gel Formulation

One example of the formula for oral hygiene composition gum gel is provided below in Table 11.

TABLE 11

| Ingredient | % w/w | Function/Use |
|---|---|---|
| BreathaLyser or | 50.00 | active product |
| BreathaLyser(+) | | |
| Purified water | 25.44 | volume |
| Glycerin 99.5% | 23.70 | surface adherent |
| Hydroxymethylcellulose gum | 0.50 | thickener |
| Polysorbate 80 | 0.25 | emulsifier |
| Barley malt syrup | 0.05 | sweetener/taste |
| | | enhancer |
| Chlorhexidine digluconate | 0.045 | preservative |
| Zinc gluconate | 0.01 | breath freshening |
| | | (binds free sulphur) |
| FD&C blue #1 | 0.005 | color |
| | 100.00 | |

### Example 9

### Compositions and Methods for Making Hard Biscuits and Cookie Treats

The ingredients for the various biscuits and cookie treats are provided in the tables below. The directions for making the biscuits and cookie treats are generally the same for all the recipes.

### Method 1

First beat the eggs and combine with other wet ingredients, mixing well. Next fold in dry ingredients to make dough, roll the dough out to approx ½ inch (˜1 cm) thick. Cut dough into appropriate sized strips or use cookie cutters. Bake the dough at 180° C. for approximately 30 minutes or at 160° C. for approximately 45 minutes. After cooking is completed, turn the oven off and allow cooling for several hours (or overnight).

Method 2 Commercial Production with Proteolytic Enzyme Emilgase

Following the steps of method 1, allow the product to cool to less than 50° C. Next spray or paint the upper surface with a concentrated enzyme solution to achieve approximately 2.5 to 5.0 mg emilgase per one day's ration of finished product. Allow the product to dry at 50° C. for at least 2 hours.

TABLE 12
Examples of Homemade Hard Biscuits or Cookie Treats

| | Finished Product |
|---|---|
| | g (oz) approx. |
| Dog Biscuit (Hard) | |
| 3.4 fl oz (100 mL) Breathalyser | |
| 0.6 fl oz (18 mL) hot water | |
| 2½ cups whole wheat flour | |
| ½ cup non-fat powdered milk | |
| 1 tsp. sugar (or less) | |
| 1 tsp. salt (or less) | |
| 1 egg, well beaten | |
| 1 tsp. beef or chicken bouillon granules | |
| | 750 (26) |
| Dog Cookies | |
| 3.4 fl oz (100 mL) Breathalyser | |
| 0.6 fl oz (18 mL) hot water | |
| ½ cup powdered milk | |
| 1 egg, well beaten | |
| 2½ cups flour | |
| ½ tsp. garlic salt | |
| 1½ tsp. brown sugar | |
| 6 tblsp. gravy | |
| 4 oz jar Baby Food (meat) | |
| | 750 (26) |
| Milk Bone Type Hard Dog Biscuits | |
| 3.4 fl oz (100 mL) Breathalyser | |
| 2.6 fl oz (75 mL) hot water | |
| ⅓ cup margarine | |
| ½ cup powdered milk | |
| 1 tsp. salt | |
| 1 egg, well beaten | |
| 3 cups whole wheat flour | |
| | 800 (28) |
| Crunchy Treats for Cats | |
| 6 fl oz (175 mL) Breathalyser | |
| 6 fl oz (175 mL) chicken or beef broth | |
| 1½ cups whole-wheat flour | |
| 1½ cups rye flour | |
| 1½ cups brown rice flour | |
| 1 cup wheat germ | |
| 1 tsp. dried kelp or alfalfa | |
| 4 tblsp. vegetable oil | |
| 1 pound ground chicken | |
| 1 to 2 tblsp. brewer's yeast | |
| | 1300 (46) |

All patents, publications and abstracts cited above are incorporated herein by reference in their entirety, including U.S. provisional patent application Ser. No. 60/532,303. It should be understood that the foregoing relates only to preferred embodiments of the present invention and that numerous modifications or alterations may be made therein without departing from the spirit and the scope of the present invention as defined in the following claims.

1-20. (canceled)

21. A method of maintaining oral health comprising administering to an animal or a human an amount of an ingestible composition comprising xylitol, a preservative, a proteolytic enzyme, and an antimicrobial agent, wherein the amount is effective to maintain oral health.

22. The method of claim 21, wherein the animal is a pet, a farm animal, or a zoo animal.

23. The method of claim 21, wherein the composition is administered in drinking water.

24. The method of claim 21, wherein maintaining oral health is treating, reducing, or preventing oral disease, gingivitis, periodontal disease, stomatitis, halitosis, plaque, tartar, calculus, or oral infection.

25. The method of claim 21, wherein administering the composition improves appearance of the oral cavity, produces whiter teeth and produces healthier gums.

26. The method of claim 21, wherein the composition is in the form of a liquid, spray, solution, paste, gel, foam, syrup, or powder.

27. The method of claim 21 wherein the composition is incorporated into food, dental chews, candies, cookies, treats, teething rings, or drinking utensils.

28. The method of claim 22, wherein the composition may be administered to the animal by an owner or caretaker of the animal.

29. The method of claim 21, wherein the composition further comprises purified water, glycerin, hydroxymethylcellulose gum, polysorbate 20, barely malt extract, chlorhexidine digluconate, zinc gluconate, dextrose, cysteine, vitamin E, sodium bicarbonate and malic acid, the preservative is potassium sorbate, and the antimicrobial agent is sodium benzoate, chlorhexidine or a chlorhexidine salt.

30. The method of claim 21, wherein the composition further comprises purified water, glycerin, polysorbate 20, chlorhexidine gluconate, zinc gluconate, the preservative is potassium sorbate, and the antimicrobial agent is sodium benzoate, chlorhexidine or a chlorhexidine salt.

31. The method of claim 21, wherein the antimicrobial agent is a benzoate, chlorhexidine or a chlorhexidine salt.

32. The method of claim 21, wherein the composition further comprises an ingredient, wherein the ingredient is an antibiotic, an antifungal, an antiseptic, an immunomodulatory, an anticancer drug, a surface adherent, a thickener, an emulsifier, a breath freshener, an anti-plaque agent or a sweetener.

33. The method of claim 21, further comprising a taste-enhancing component.

34. The method of claim 21, wherein the animal is an amphibian, reptile, aves, mammal, marsupial, hoofed animal, sheep, pig, giraffe, zebra, carnivore, marine animal, primate, feline, dog, cat, rabbit, guinea pig, hamster, gerbil, ferret or horse

35. The method of claim 21, wherein the composition is in the form of a toothpaste, lozenge, lollipop, tablet, flash melt formulation, gum, candy, coating on a teeth whitening strip, coating on dental floss, coating on a dental mould, orthodontic device, a chew toy, rawhide chew, a dog biscuit, carbonated beverage, bottled water, flavored water, sports drink, juice nutritional supplement, animal feed, a pet treat, or pet food.

36. A method of maintaining oral health comprising administering to an animal or a human an amount of an ingestible composition comprising purified water, glycerin, xylitol, polysorbate 20, chlorhexidine gluconate, potassium sorbate, a proteolytic enzyme, zinc gluconate and sodium benzoate, wherein the amount is effective to maintain oral health.

37. The method of claim 36, wherein the proteolytic enzyme is present in the composition in an amount of from about 0.001% to about 50%, from about 0.001% to about 10%, from about 0.005% to about 5.0%, from about 0.01% to about 2.5% or from about 0.02% to about 2.0% on a weight/weight basis.

38. The method of claim 21, wherein the proteolytic enzyme comprises an enzyme derived from a species of bacteria from the genus Bacillus.

39. The method of claim 36, wherein the proteolytic enzyme comprises an enzyme derived from a species of bacteria from the genus Bacillus.
